(19) United States
(12) Patent Application Publication
(10) Pub. No.: US 2021/0330900 A1
(43) Pub. Date: Oct. 28, 2021

(54) TARGETED ADMINISTRATION TO THE OROPHARYNX OF VISCOUS OR DRY POWDER FLUTICASONE PROPIONATE AND RELATED CORTICOSTEROIDS FOR THE TREATMENT OF EOSINOPHILIC ESOPHAGITIS (EoE)

(71) Applicant: Topical Sinus Therapeutics, Inc., Camarillo, CA (US)

(72) Inventors: Russell N. Reitz, Camarillo, CA (US); John C. Tarrant, Camarillo, CA (US)

(73) Assignee: Topical Sinus Therapeutics, Inc., Camarillo, CA (US)

(21) Appl. No.: 17/238,045

(22) Filed: Apr. 22, 2021

Related U.S. Application Data
(60) Provisional application No. 63/014,060, filed on Apr. 22, 2020.

(51) Int. Cl. A61M 11/00; A61K 31/566; A61K 31/58; A61K 31/57; A61K 31/573; A61K 31/55; A61K 31/335; A61K 31/138; A61K 31/4545

(52) U.S. Cl. CPC A61M 11/008; A61K 31/566; A61K 31/58; A61K 31/57; A61M 2210/065; A61K 31/55; A61K 31/335; A61K 31/138; A61K 31/4545; A61K 31/573

(57) ABSTRACT

Embodiments of the application involve a formulation of Fluticasone Propionate in the form of a suspension, gel or dry powder comprising Fluticasone Propionate, Fluticasone Furoate, Micronized Fluticasone Propionate or Micronized Fluticasone Furoate or other corticosteroids. Additional pharmaceutical components of the formulation may allow for the adherence and duration of the active ingredient to the inflamed esophageal tissues. The resulting medication formulation is then targeted with a drug delivery device and applied directly to the oropharynx bypassing the oral cavity altogether. The medication then flows down the back of the throat to the esophagus. By passing the oral cavity prevents the corticosteroid drug from being absorbed into the bloodstream through oral cavity tissues, helping to prevent harmful side effects. Additional side effects are avoided by using the medication Fluticasone, which exhibits exceptionally low gastrointestinal absorption.

### RELATED APPLICATIONS

This application claims priority to U.S. Provisional Patent Application No. 63/014,060 filed on Apr. 22, 2020, the content of which is incorporated herein by reference in its entirety.

### TECHNICAL FIELD

The present disclosure relates generally to targeted drug administration, and more specifically, some embodiments relate to the targeted topical administration of corticosteroid formulations for the treatment of eosinophilic esophagitis (EoE). In particular, some formulations are found in powder, suspension, or gel form.

### BACKGROUND

Eosinophilic esophagitis (“EoE”) is an allergy of the esophagus brought about by a patient's allergy to certain foods, various pollens, and other substances such as molds, dust mites and animal dander. EoE is an allergic inflammatory condition, a chronic disease affecting the esophagus, the muscular tube that carries food from the mouth to the stomach. The allergy involves the infiltration of eosinophils (a certain type of white blood cell that elevates in tissues responding to the allergy). In response to the allergy, eosinophils increase in the tissues and release substances into surrounding tissues that cause inflammation. Eosinophils are normally not found in esophageal tissues. Further research suggests a specific protein found in esophageal tissues of healthy individuals is nearly absent in those same tissues of patients diagnosed with EoE. This protein is thought to decrease inflammation in the tissues lining the esophagus and protect esophageal tissues from chemicals and/or enzymes found in certain foods.

The tissue infiltration of eosinophils is indicative of tissue damage leading to the following symptoms in adults: heartburn; continued reflux not improved by medications; trouble swallowing food; chest pain; food getting stuck in the esophagus; regurgitation of food; and upper abdominal pain. In addition, eosinophils result in the tissue damage leading to the following symptoms in children: abdominal pain; vomiting; poor appetite; trouble swallowing; reflux not improved by medications; and difficulty eating.

To confirm EoE, an upper gastrointestinal endoscopy is performed. The procedure involves placing a flexible tube through the mouth down into the esophagus. The tube contains a light and camera at the end allowing the physician to visually inspect the esophageal tissues. The tissue may show signs of narrowing, spots, inflammation, and horizontal rings. During the endoscopy, the physician will take tissue samples to be tested for infiltration of eosinophils confirming the diagnosis of EoE.

There is no current cure for eosinophilic esophagitis. This allergy induced, auto immune disease, afflicts approximately 1 out of every 2500 individuals; however, this estimate may be extremely low due to underreporting of EoE as confusion may still exist with gastroesophageal reflux disease (GERD) and EoE symptoms that may change over time. The prevalence of EoE in males is greater than in the female population.

As set forth below, existing treatments include the use of proton pump inhibitor (PPI's) drugs, corticosteroids, and the use of specific diet restrictions.

Proton pump inhibitors are a class of medications. The proton pump chemical pathway is found in the cells lining the stomach, parietal cells, that make gastric acid. PPIs inhibit the activity of the proton pump decreasing gastric acid secretion. This class of drugs prevent gastric acid secretion; they do not lesson the allergic reaction causing EoE. In some patients PPI's may also reduce esophageal tissue inflammation. Long term side effects of using a PPI may include kidney disease, infections, and fractures. Common side effects may include nausea, vomiting, diarrhea, and headaches.

Corticosteroids comprise a large class of medications, including but not limited to triamcinolone, beclomethasone, methylprednisolone, prednisolone, prednisone, betamethasone, ciclesonide, mometasone, fluticasone, budesonide, cortisone, and hydrocortisone. Corticosteroids work by limiting the activity of the immune system. Inflammation, the process in which the body's white blood cells, specifically eosinophils, infiltrate the esophageal tissues. One current off-label treatment includes swallowing a viscous budesonide drug preparation. The drug budesonide was FDA approved for the treatment of pulmonary tissue inflammation applied by inhaling an aerosolized suspension of this drug. Another off-label treatment may include a fluticasone propionate pulmonary inhaler. This oral drug inhaler although approved for pulmonary inhalation through the oral cavity is also prescribed for an off-label treatment of EoE. In this case the patient is instructed to apply 1-2 puffs of dry powder inside their cheeks, swirl the powder with saliva by the tongue, and then swallow the saliva drug mixture in an attempt to coat the esophagus. Because of residual buccal tissue absorption, patients are instructed to rinse and spit following the dose.

Dietary restrictions for the treatment of EoE include a six-food elimination diet. The diet typically trials the exclusion of nuts, wheats, soy, eggs, milk, (fish and shellfish). The food trials are set to identify and remove a food allergy trigger of EoE from the diet.

In view of the above, there exists an urgent unmet need for an effective treatment for EoE which addresses the current drug therapy issues including: (1) Eliminating oral cavity absorption. (2) Eliminating the need for systemically absorbed oral drug tablet/capsule therapy. (3) Creating a system for the targeted topical administration of drug near the site of inflamed tissues, bypassing the oral cavity. (4) Creating an extended duration of time the drug may adhere to the inflamed esophageal tissues.

### SUMMARY

The present application is directed towards targeted drug administration of a suspension of micronized fluticasone propionate, a gel incorporating fluticasone propionate, or a microfine dry power of fluticasone propionate. The quantity, concentration and volume of the drug may vary. During administration, a specific dose/volume is applied to the back of the oropharynx.

In some embodiments, the fluticasone propionate may be applied onto the tissues of the oropharynx using an applicator tip extension device that bypasses the tongue and oral tissues with the applicator located at the back of the mouth on top of the tongue. This device may also be held in place securely by biting down on the device itself while depressing an actuator, trigger, plunger, syringe, or metered dose applicator connected to a volume of medication.

### BRIEF DESCRIPTION OF THE DRAWINGS

FIG. 1 illustrates a delivery device for bypassing a patient's oral cavity and delivering a medicated formulation to inflamed esophageal tissues, in accordance with an embodiment of the application.

FIGS. 2 and 3 illustrate vials 170, 180 for containing the medicated formulations described herein, in accordance with embodiments of the application.

FIG. 4 illustrates a delivery device comprising a squeeze apparatus 230 for delivering the medicated formulations described herein, in accordance with embodiments of the application.

FIGS. 5 and 6 illustrate additional delivery devices comprising curved catheters 210, 240 for delivering the medicated formulations described herein, in accordance with embodiments of the application.

FIG. 7 illustrates a further delivery device 290 for delivering the medicated formulations past the oral cavity to the back of the throat described herein, in accordance with embodiments of the application. The delivery device 290 includes holes 295 in the distal end of the applicator to allow for the passage and delivery of the medicated formulations.

FIGS. 8 and 9 illustrate additional delivery devices 270, 280 for delivering the medicated formulations described herein, wherein the dose volume is connected to an atomizer type spray apparatus, in accordance with embodiments of the application.

FIG. 10 illustrates another delivery device comprising a winged device attached to a catheter for delivering the medicated formulations described herein, in accordance with embodiments of the application.

FIG. 11 illustrates a metered dose delivery device 300 for delivering the medicated formulations over the tongue to the throat, in accordance with embodiments of the application.

### DETAILED DESCRIPTION OF THE EMBODIMENTS

Embodiments of the invention are directed toward a formulated suspension, gel or dry powder of micronized fluticasone propionate or other corticosteroid in a volume of 1 cc to 30 ccs. Included in a formulation are excipient components which will increase micronized particle solubility, viscosity, and adherence of the formulation to the inflamed esophageal tissues.

In one embodiment, the active pharmaceutical ingredient is micronized fluticasone propionate. A selected quantity of the micronized drug is added to a quantity of sterile water to provide for the formula batch concentration and total volume. While stirring the drug-sterile water mixture quantities of non-active drug ingredients are added to increase viscosity and solubility of the formula. Additional non-active ingredients, such as various polymers may be added allowing greater adherence and layering of the suspension onto the affected tissues.

While continuing to stir the final formulation transfer the final volume into a clean container such as a 1000 cc to 3000 cc Pyrex beaker. The final formulation is then homogenized further reducing particle size (solid in liquid) using various available rotor-blade homogenizers mounted on a pole with stand.

Once homogenization is complete the drug suspension is transferred into various devices for dispensing. Such dispensing devices can include but are not limited to: (1) plastic or glass medication vials are filled with volumes ranging from 1 cc to 15 cc; (2) plastic luer-lock syringes are filled with volumes ranging from 1 cc to 30 cc; and (3) plastic bottles ranging in size from 60 cc to 240 cc with spray actuator and catheter included.

Embodiments of the invention involve creating a suspension of micronized fluticasone that is applied directly onto the esophagus. This may be done by swallowing the drug making as little contact with oral tissues as possible or by spraying the drug directly onto the esophagus with a device that by-passes the tongue and oral tissues altogether.

Fluticasone propionate is a corticosteroid used to treat various types of upper and lower respiratory inflammatory diseases. By way of example, a fluticasone propionate metered dose inhaler is prescribed to treat pulmonary inflammation. Some embodiments of the present application involve a formulation of fluticasone propionate in the form of a suspension comprising fluticasone propionate, fluticasone furoate, micronized fluticasone propionate or micronized fluticasone furoate with additional pharmaceutical components added, which allow for the adherence of the active ingredient to the inflamed esophageal tissues. The use of fluticasone is particularly relevant. Unlike other corticosteroids, Fluticasone exhibits exceptionally low gastrointestinal absorption with 99% of the drug inactivated by 1st pass hepatic metabolism into a single inactive metabolite. Thus the risk of potential side effects are significantly reduced,

In some embodiments, the formulation can be swallowed in a specific dose volume.

In other embodiments, the formulation may be sprayed via various devices in a specific dose volume bypassing the oral cavity, such that the formulation will slowly descend from the upper throat coating the inflamed esophageal tissues.

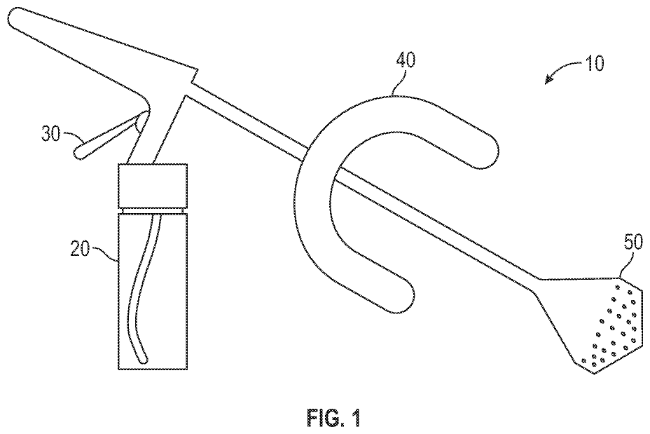

Referring to FIG. 1, a delivery device 10 is depicted for bypassing a patient's oral cavity and delivering the formulation to the inflamed esophageal tissues. Specifically, the delivery device includes a medication bottle 20 containing the formulation and in fluid communication with a trigger 30, a mouthpiece 40 and an atomizer 50. In operation, the user inserts the atomizer 50 into her mouth adjacent the back of the upper throat, and then bites down on the mouthpiece 40. Next, the user squeezes the trigger 30 to pressurize the formulation, thereby forcing it out of the atomizer 50 in the form of a fine spray. Once the formulation is sprayed on the upper throat, it slowly descends down the throat and coats the inflamed esophageal tissues. In some embodiments, the delivery device 10 can be adapted to spray a predetermined amount of formulation when the trigger 30 is squeezed.

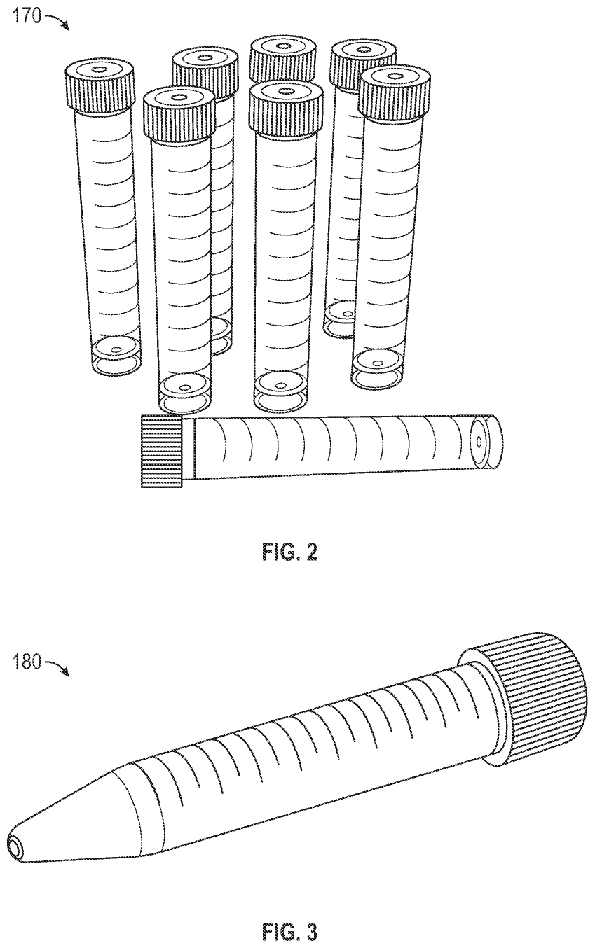

Referring to FIGS. 2 and 3, vials for containing the formulations described herein. The vials are sized to contain a prescribed dose of the formulation to be swallowed by a patient or poured into a syringe or other drug delivery device providing a targeted application to the back of the throat. In some embodiments, the formulation may comprise the active ingredient, fluticasone propionate.

In other embodiments, the formulation may comprise the active ingredient micronized fluticasone propionate.

In further embodiments, the formulation may comprise the active ingredient fluticasone furoate.

According to additional embodiments, the medicated formulation may comprise the active ingredient micronized fluticasone furoate.

In various embodiments of the present application, the formulation may include other corticosteroids such as: Budesonide, Beclomethasone, Betamethasone, Ciclesonide, Mometasone Furoate Monohydrate, Mometasone Furoate, Hydrocortisone, Cortisone, Prednisone, Prednisolone, Methylprednisolone, Dexamethasone and Triamcinolone.

One embodiment includes a suspension in a volume between 1 cc to 30 cc's. One embodiment includes a gel volume between 1 cc to 30 ccs. An additional embodiment includes a micronized dry power expelled in a metered dose to range from 0.05 milligrams to 10 milligrams per dose.

In further embodiments, the formulation may include other medications such as antihistamines such as azelastine, olopatadine, diphenhydramine, loratadine and/or other antihistamines.

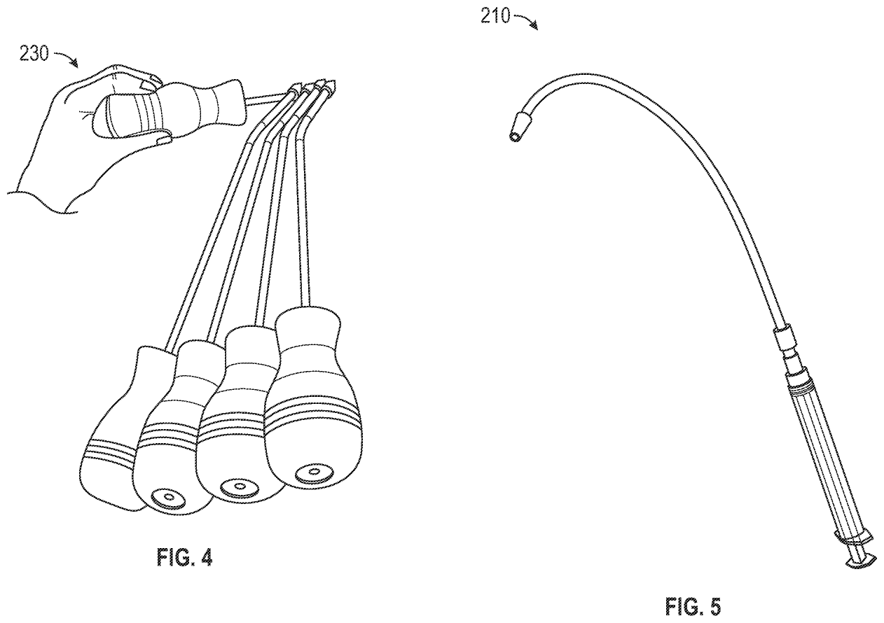

According to another embodiment, the dose volume may be connected to a delivery device in the form of a squeeze apparatus, such as illustrated in FIG. 4.

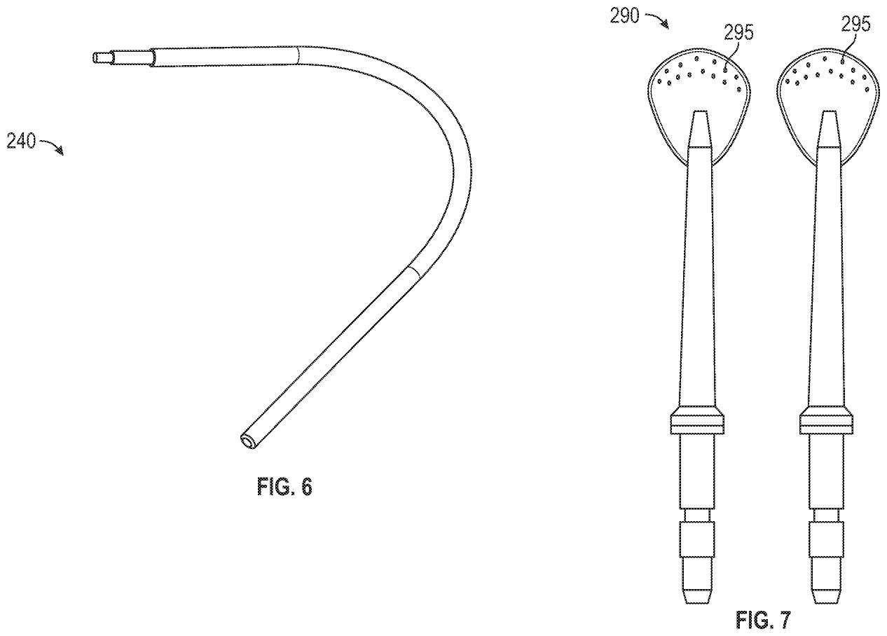

Some embodiments may include a curved catheter (FIGS. 5 and 6) or with various bends to deliver the dose to the oropharynx bypassing the oral cavity. One such embodiment may include a silicone catheter (FIG. 5) or other polymer material (FIG. 5). Another embodiment may include a metal catheter (FIG. 6) with flexible bends allowing specific placement of drug to reach the inflamed tissues.

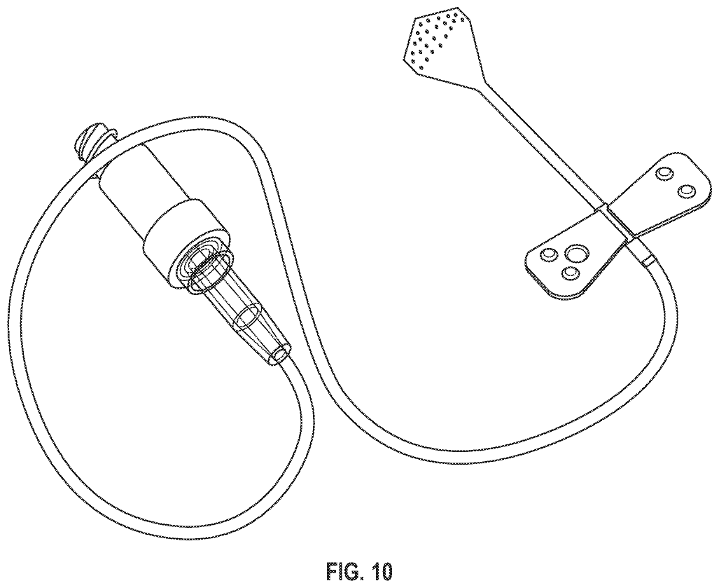

Referring to FIG. 10, another embodiment comprises a winged device attached to a metal catheter, silicone catheter or another specific polymer. The winged device may be moved up and down the length of the catheter so that the end tip position distance may be adjusted and held in place by the patient biting down on the winged device.

According to additional embodiments, the dose volume is connected to a spray apparatus with a catheter to deliver the dose to the oropharynx, thereby bypassing the oral cavity. As an example, a 1 cc to 3 cc volume per actuation is developed.

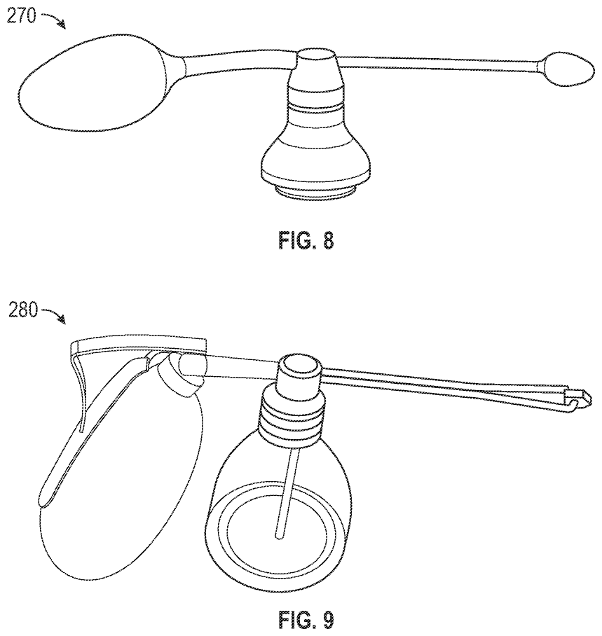

In some embodiments, the tip of the catheter creates a spray ranging from 1 to 180 degrees. One such embodiment (FIG. 8) may include the tip of the catheter having a bulb spray configuration that allows the spray to coat a 360-degree range to cover the inflamed tissues. Another such embodiment (FIG. 6) may include the tip of the catheter to be made from a silicone polymer, other related flexible polymer, or metal composite material.

FIG. 10 illustrates another delivery device in accordance with a further embodiment. In operation, the patient bites down on the wing to hold the device in place. A tube or catheter extends to the back of the patient's mouth past the tongue, near the esophagus, with a directional spray device attached at the distal end for spraying the oropharynx when a pump is activated. In the illustrated embodiment, a hand pump is disposed on proximal end of device, such that when squeezed, medication is sprayed from the spray device. The device can include a wing or mouthpiece for the patient to bite down on, such as described with respect to FIG. 1.

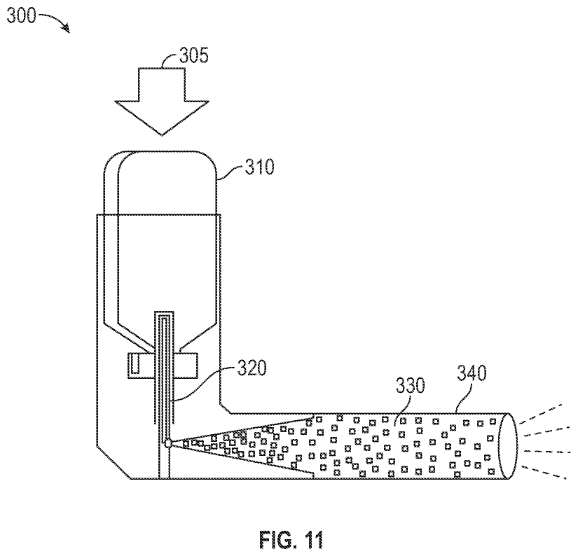

FIG. 11 illustrates another delivery device according to yet another embodiment. In particular, FIG. 11 depicts a triggered device apparatus 300 comprising an oral metered dose applicator with a catheter extension. In operation, a patient applies a force indicated by arrow 305 to a canister 310 containing the medication formulation to trigger a metered dose of the medication formulation to flow through metering dose valve 320 and be delivered to the targeted area in the form of a spray of particles 330. Specifically, the spray flows through an extension tube 340 configured to bypass the patient's oral cavity (over the tongue) for delivery of the medicated formulation to the patient's oropharynx region.

Further embodiments may include various medical mucoadhesives, such as chitosan (and chitosan derivatives), or other tissue adhesive polymers such as carboxymethylcellulose, poly acrylic acid, carbopol, hyaluronic acid, polycarbophil, polylactide and pl-co glycolide.

Additional embodiments may include viscosity enhancers, such as the cellulose polymers, namely: (1) methyl cellulose, (2) hydroxyethyl cellulose, (3) carboxymethyl cellulose, (4) hydroxypropyl methylcellulose, (5) sodium carboxymethyl cellulose and (6) hydroxypropyl cellulose.

Some embodiments may include humectants such as, glycerin, sorbitol, propylene glycol, aloe vera, and xylitol.

Further embodiments may include sterile water, purified water, or distilled water.

Other embodiments may include a sweetening agent such as sodium saccharin.

Additional embodiments may include a preservative such as calcium EDTA, disodium EDTA, benzyl alcohol or benzalkonium chloride.

Some embodiments may include various saline concentrations, hypertonic, hypotonic, or isotonic.

Further embodiments may include the use of various buffering agents such as citric acid, sodium citrate, phosphoric acid, sodium bicarbonate, boric acid, mono sodium phosphate and disodium phosphate.

Additional embodiments may include the use of various surfactants such as polysorbate 60, polysorbate 80 or polysorbate 20.

Some embodiments may include the use of flavoring agents such as natural grape, cherry, vanilla, or orange flavoring.

Further embodiments may include the use solubility enhancers such as alpha, beta cyclodextrin and other cyclodextrin complexes.

Various embodiments have been described with reference to specific exemplary features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the present application, whether or not such embodiments are described and whether or not such features are presented as being a part of a described embodiment. Thus, the breadth and scope of the present application should not be limited by any of the above-described exemplary embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary diagrams and other illustrations. As will become apparent to one of ordinary skill in the art after reading this document, the illustrated embodiments and their various alternatives can be implemented without confinement to the illustrated examples. For example, diagrams and their accompanying description should not be construed as mandating a particular configuration.

What is claimed is:

1. A therapy for the treatment of a patient's inflamed esophageal tissues, comprising:
a medicated formulation; and
a delivery device for bypassing the patient's oral cavity and delivering the medicated formulation directly to the oropharynx and indirectly to the inflamed esophageal tissues.

2. The therapy of claim 1, wherein the medicated formulation is selected from the group consisting of fluticasone propionate, fluticasone furoate, micronized fluticasone propionate and micronized fluticasone furoate.

3. The therapy of claim 1, wherein the medicated formulation comprises budesonide.

4. The therapy of claim 1, wherein the medicated formulation is selected from the group consisting of triamcinolone, beclomethasone, methylprednisolone, prednisolone, prednisone, betamethasone, ciclesonide, mometasone, cortisone, dexamethasone, and hydrocortisone.

5. The therapy of claim 1, wherein the medicated formulation includes an antihistamine selected from the group consisting of azelastine, olopatadine, diphenhydramine and loratadine, wherein the medicated formulation does not contain a corticosteroid.

6. The therapy of claim 1, wherein the medicated formulation comprises a viscous suspension containing fluticasone propionate, fluticasone furoate, budesonide, triamcinolone, beclomethasone, methylprednisolone, prednisolone, prednisone, betamethasone, ciclesonide, mometasone, cortisone, dexamethasone, or hydrocortisone, wherein the delivery device comprises a syringe with catheter, a squeeze apparatus, a spray apparatus, a catheter or a metered dose applicator targeting the oropharynx.

7. The therapy of claim 1, wherein the medicated formulation is selected from the group consisting of fluticasone propionate, fluticasone furoate, micronized fluticasone propionate and micronized fluticasone furoate, and wherein the delivery device comprises a squeeze apparatus, a spray apparatus, or a catheter.

8. The therapy of claim 1, wherein the medicated formulation comprises a dry powder containing fluticasone propionate, fluticasone furoate, budesonide, triamcinolone, beclomethasone, methylprednisolone, prednisolone, prednisone, betamethasone, ciclesonide, mometasone, cortisone, dexamethasone, or hydrocortisone, wherein the delivery device comprises a squeeze apparatus, a spray apparatus, or a metered dose applicator targeting the oropharynx.

9. The therapy of claim 1, wherein the medicated formulation is targeted to the oropharynx region of the patient.

10. The therapy of claim 1, wherein the delivery device comprises a means for applying a targeted topical dose of the medicated formulation to the oropharynx region of the patient by bypassing the patient's oral cavity.

11. The therapy of claim 1, wherein the medicated formulation comprises a viscous gel containing fluticasone propionate, fluticasone furoate, budesonide, triamcinolone, beclomethasone, methylprednisolone, prednisolone, prednisone, betamethasone, ciclesonide, mometasone, cortisone, dexamethasone, or hydrocortisone, wherein the delivery device comprises a syringe with catheter, a squeeze apparatus, a spray apparatus, a catheter or a metered dose applicator targeting the oropharynx.
